(19) United States
(12) Patent Application Publication
(10) Pub. No.: US 2021/0068669 A1
(43) Pub. Date: Mar. 11, 2021

(54) Systems And Methods For Prediction Of Glycemia And Decisions Support

(71) Applicant: HILLO AI, Palaiseau (FR)

(72) Inventors: Stéphane Bidet, Issy-Les Moulineaux (FR); Nicolas Caleca, Saclay (FR); Mikael Rehn, Paris (FR); Lucas De La Brosse, Paris (FR); Thibault Camalon, Paris (FR)

(73) Assignee: HILLO AI, Palaiseau (FR)

(21) Appl. No.: 16/958,807

(22) PCT Filed: Dec. 28, 2018
(86) PCT No.: PCT/EP2018/097088

§ 371 Date: Jun. 29, 2020

(30) Foreign Application Priority Data
Dec. 29, 2017 (EP) 17306971.7

(51) Int. Cl. A61B 5/0205; A61B 5/145; A61B 5/00; A61B 5/11; A61B 5/16; A61B 5/0402; A61B 5/0476; G16H 20/17; G16H 10/40; G16H 50/20; G16H 50/50; G06N 3/04; G06N 3/08

(52) U.S. Cl. CPC A61B 5/0205; A61B 5/02416; A61B 5/14546; A61B 5/7275; A61B 5/7264; A61B 5/1118; A61B 5/165; A61B 5/0402; A61B 5/0476; A61B 5/4848; A61B 5/486; A61B 5/742; G16H 20/17; G16H 10/40; G16H 50/20; G16H 50/50; G06N 3/04; G06N 3/08; A61B 5/14532

(57) ABSTRACT

Method for predicting glycemia of a patient including the steps of: Retrieving a data set (2) of dated glycemia measured up to the time t on the patient; Retrieving a quantity of insulin (5) injected in the patient; Retrieving a quantity of glucose (6) ingested by the patient; Generating, by a pharmacokinetic pre-processing device (20), an insulin blood concentration signal (21) and a carbs rate of appearance signal (22), from the glycemia data set (2), the quantities of injected insulin (5) and ingested glucose (6), Predicting by at least one Neural Network at least one glycemia (41) at prediction time t+Δ\hat{t} from the glycemia data (2) and from the signals generated (21) (22), the neural network being a Recurrent Neural Network able to keep in memory at each iteration information from any previous iteration.

### TECHNICAL FIELD

The technical field of the present invention relates to systems and methods for predicting glycemia, namely glucose or sugar concentration in the blood of a subject.

More particularly, the subject-matter disclosed herein relates to predictions of glycemia employing multiple parameters including but not limited to blood glucose concentration measurements, insulin treatment data, nutritional intake and physical activity measurements.

### BACKGROUND OF THE INVENTION

Different populations may benefit from a system and a method for predicting glycemia: patients with diabetes under insulin therapy, patients with diabetes under other medication and also non diabetic people.

Patients with diabetes under insulin therapy can be patient with a type 1 or a type 2 diabetes. Predict their blood sugar level can help them assess the correct doses of insulin to inject, the correct amount of carbs to ingest, and anticipate risks of hypoglycemia or hyperglycemia.

Patients with diabetes under other medication than insulin are patient with type 2 diabetes. Predict their blood sugar level can help them anticipate risks of hypoglycemia.

Predict the blood sugar level of non diabetic people can be of interest for athletes or people interested in achieving physical performances. An insulin peak associated with a postprandial blood glucose could cause a drop in performance, a low blood sugar level could cause a loss of concentration. Predict the blood glucose will help anticipate these events and act consistently to prevent them (ingestion of optimal amount of carbs, adjustment of nutrients, etc. . . . ).

Diabetes mellitus is a group of long-term metabolic disorders characterized by high blood sugar concentrations.

It is one of the largest global health emergencies.

According to the International Diabetes Federation, more than 415 million adults are estimated to currently have diabetes worldwide, and there are 318 million adults with impaired glucose tolerance, which puts them at high risk of developing the disease in the future.

There are two main types of diabetes mellitus. Type 1 diabetes results from the pancreas failure to produce insulin because of beta-cells destruction. Type 2 diabetes results from cell failure to respond properly to insulin, and can also lead to a lack of insulin.

As insulin regulates the metabolism of carbohydrates, fats and proteins by promoting the absorption of glucose from the blood into the cells, a lack of insulin leads to an inability to control blood glucose concentration.

When blood sugar concentration gets too low (hypoglycemia), it may result in a variety of symptoms including loss of consciousness, seizures or death. On the other hand, high blood sugar levels (hyperglycemia), chronically and for extended periods of time, can lead to serious complications, including kidney damage, neurological damage, cardiovascular damage, vision loss and peripheral neuropathy.

Therefore, to prevent serious complications, it is vital for many diabetic patients to self-administer exogenous insulin and ingest carbohydrates in order to maintain normal blood glucose concentrations. Through this kind of operation, patients are required in a sense to emulate the pancreas.

Unlike many medicines, insulin cannot be taken orally, it is usually taken as subcutaneous injections by single-use syringes or repeated-use insulin pens with needles, or by a continuous subcutaneous insulin infusion pump. In all cases, several times each day, patients must assess insulin doses based on blood glucose measurements.

Assessing the appropriate dosing of insulin is the main difficulty experienced by patients with diabetes: it is highly complex since there are many parameters which influence blood glucose concentration, including but not limited to: amount of insulin still present and active in the body, past and upcoming nutritional intakes, physical activities, emotional states, sleep quality and patterns. Moreover, the effect of those factors on subsequent glucose levels has been observed as patient specific.

Similarly, patients must deal with low blood sugar emergencies, usually by eating something that contains sugar.

Managing those situations requires precautions: for instance, patients should not drive if their blood glucose concentration is too low, they must anticipate physical activities as exercise is a common stressor causing perturbation to glycemia, with diverging effects according to the exercise type (aerobic or anaerobic).

Anticipating low blood sugar emergencies is also another difficulty: it is as complex as assessing an insulin dosage because of the multitude of parameters which influence glucose concentrations.

Different methods for diabetes management exist: flexible insulin therapy (FIT) helps patients assess insulin doses proportionally to nutritional load of the meal, for example estimated amount of carbohydrates.

Even if FIT allows a better glycemic control, it has clear limitations, such as considering only one of the factors involved in blood glucose fluctuations. It also cannot help the patients to anticipate low blood sugar emergencies.

To improve glycemic control and avoid future occurrences of hyperglycemia and hypoglycemia, there is a need of a system and a method able to produce more reliable prediction, and to present convenient recommendations to patients accordingly.

The recent advent of continuous glucose monitoring (CGM) technology, which gives frequent measurements (e.g. every 5 minutes) of the glucose level in the subcutaneous tissue where the sensor is placed paved the way to new approaches since research showed that physiological datasets patterns exist in relation to blood glucose concentration fluctuations.

Numerous attempts to model the glucose-insulin system can be found in prior art (see, e.g. Balakrishnan et al. (2011) Review and analysis of blood glucose (BG) models for type 1 diabetic patients. Industrial & Engineering Chemistry Research. 50 (21), pp. 12041-12066). These models are knowledge-driven models based on physiology or data-driven models.

Knowledge-driven models are qualitative physiological models wherein inputs are run on an explicit mathematical description of the body's metabolism.

The most recognized and sophisticated knowledge-driven model was developed by Dalla Man (see Dalla Man et al. (2007) Meal Simulation Model of the Glucose-Insulin System, IEEE Transactions on Biomedical Engineering. 54, pp. 1740-1749). This model includes 35 parameters and 13 state variables.

To use such a model as a glycemia fluctuations predictor for one specific patient with diabetes, many parameters must be evaluated for this patient, which require complex clinical trials.

Moreover, this kind of knowledge-driven model is necessary incomplete since it takes into account a limited number of parameters and states variables. It cannot thereby model all biological mechanisms related to glycemia: for instance, the model developed by Dalla Man does not take into account the physical activity of the patient.

Data-driven models simply relate the input to the output. Those models have been investigated since the advent of CGM technology.

They started with Auto-Regressive (AR) time series modelling, and then exogenous input were included (see, e.g. Van Herpe et al. (2006) An adaptive input-output modeling approach for predicting the glycemia of critically ill patients. Physiological Measurement. 27 (11), pp. 1057-1069. Or Finan et al. (2008) Identification of empirical dynamic models from type 1 diabetes subject data. Proceedings of the American Control Conference, Seattle. pp. 2099-2104).

More recently, feed-forward neural network models have been shown to be a competitive approach against ARX models, which are evolutions of AR time series modelling (see Daskalaki et al. (2012) Real-time adaptive models for the personalized prediction of glycemic profile in type 1 diabetes patients. Diabetes Technology & Therapeutics. 14 (2), pp. 168-74).

Diabetes mellitus system is extremely complex in nature: it is dynamic, non-linear, interactive and patient-specific. Artificial Neural Network (ANN) and Recurrent Neural Network (RNN) are approaches gaining considerable interest due to their inherent natures which are well suited to model complex physiological systems (see, e.g. Zecchin et al. (2012) Neural network incorporating meal information improves accuracy of short-time prediction of glucose concentration. IEEE Transactions on Biomedical Engineering. 59 (6), pp. 1550-1560).

A Recurrent neural network (RNN) is made up of repeating feed-forward neural networks connected in series.

Each feed-forward neural network has its own output, and its input is concatenated with the output of the previous feed-forward neural networking the chain.

This structure allows RNN to handle a sequence of inputs of any length and to pass information between states in an input sequence.

Therefore, RNN are very interesting for working with sequential and dynamic data such as blood glucose level time series.

More generally, RNN are used when past information is needed to perform the present task, as for instance trying to predict the next word of a sentence based on the previous ones.

But when a lot more of context is needed, when the gap between the relevant information and the point where it is needed is very large, RNNs become unable to learn to connect the information and fails to learn dependencies.

This is particularly relevant in the case of glycemia prediction where many multi-dependent chemical and physical quantities are involved.

That is why there is need for a new tool overpassing the limitation of traditional RNNs.

In a recent study, the approaches of a Knowledge-Driven model and a Data-driven model were combined. A physiological model aimed at better capturing the dynamic between blood glucose and daily events was introduced in a Support Vector Regression (SVR) Model.

This new data model turns out to improve the prediction accuracy over older models (see Bunescu et al. (2013) Blood glucose level prediction using physiological models and support vector regression. Machine Learning and Applications, 12^{th} International Conference. 1, pp. 135-140).

With more than 1400 days of clinical patient data, their adaptive model for glycemia prediction manage to get an error of 19% compared to the true value for a prediction horizon of 30 to 60 minutes. The error is evaluated by the average ratio of absolute difference between the predicted glycemia and the temporally corresponding measured glycemia divided by the measured glycemia.

The performance of a prediction model can also be measured using the Root Mean Square Error (RMSE) which is the square root of the average squared difference between the predicted glycemia and the temporally corresponding measured glycemia.

Other parameters used to evaluate the performance of a prediction model are the Mean Absolute Difference (MAD) and the Mean Absolute Relative Difference (MARD) which are traditionally used to assess the accuracy of continuous glucose monitoring systems.

MAD is the average absolute value of the difference between the predicted glycemia and the temporally corresponding measured glycemia.

MARD is the average absolute value of the difference between the predicted glycemia and the temporally corresponding measured glycemia divided by the temporally corresponding measured glycemia.

Another option to measure the performance of a prediction model is a Clark Error Grid Analysis (EGA).

Parkes Error Grid Analysis may also be used. This is a standardized metric to measure the performance of CGM signals in relation to reference measurements (see J. L. Parkes, S. L. Slatin, S. Pardo, B. H. Ginsberg. A new consensus error grid to evaluate the clinical significance of inaccuracies in the measurement of blood glucose. Diabetes Care, 23(8):1143-1148, 2000). In several previous studies one or two of those error metrics were used.

Considering the permitted error of commercially used devices of 15% and rapid insulin peak effect in 90 minutes, further development is required.

Focusing on the identification of additional types of signals to increase complexity of physiological models would be very time-consuming.

Combining a more complex physiological model and a powerful Neural Network may lead to better predictions, but also to more important data collection, more and longer training periods, and longer calculation times to obtain a prediction.

To increase prediction accuracy, there is a need to explore alternative approaches.

In view of the above, methods offered to date do not satisfy patient needs.

There is a need to offer to diabetic patients a glycemia prediction system featuring shorter data collection, fewer and shorter training periods, shorter calculation time to predict a glycemia level and greater accuracy for example an error of 10 mg/dl for a 30 minutes prediction and 20 mg/dl for a 90 minutes prediction time, or also for example MARD below 10% for a 30-minutes prediction, below 20% for a 60-minutes prediction and below 25% for a 90-minutes prediction horizon, and more than 99% of predictions within zones A and B using a Parkes EGA for 30-minutes prediction horizon.

Furtherly, those features get even more demanding for a glycemia decision support system which implies a recursive use of the prediction model, especially when it is implemented for the real-time use of tens of thousands diabetic patients sending simultaneous requests to the system.

Due to vast computational and data resources required to regularly train patient-centered models and to recursively use them in real time, a deployment of the solution for tens of thousands of users at the same time implies to develop and tune specific elements for each of the methods for the overall process to work with the desired level of safety, accuracy and robustness.

### PRESENTATION OF THE INVENTION

The object of the invention is to overcome the problems mentioned herein before.

The object of the invention is a method for predicting glycemia in a subject including the steps of:
Retrieving a data set of dated glycemia measured up to the time t on the patient;
Retrieving a quantity of insulin injected in the patient;
Retrieving a quantity of glucose ingested by the patient;
Generating, by a pharmacokinetic pre-processing device, an insulin blood concentration signal and a carbs rate of appearance signal, from the glycemia data set, the quantities of injected insulin and ingested glucose,
Predicting by at least one Neural Network at least one glycemia at prediction time t+Δt from the glycemia data and from the signals generated, the neural network being a Recurrent Neural Network able to keep in memory at each iteration information from any previous iteration.

The method for predicting glycemia may be applied to any patient with diabetes, treated with insulin or not, or to any non-diabetic people. In the case of non-diabetic people or patient without insulin therapy, the quantity of insulin injected in the patient would of course set to zero. That does not raise any obstacle in successfully running the method.

The method may be appropriately completed by the following steps, alone or in any of their technically possible combinations:
Retrieving at least one type of food ingested by the subject and at least one corresponding quantity of food ingested, the type of food being chosen among lipids, free fatty acids, proteins and fibers;
Generating, by the pharmacokinetic pre-processing device, an insulin blood concentration signal, a carbs rate of appearance signal and optionally a free fatty acid blood concentration signal, from the glycemia data, the quantities of injected insulin and ingested glucose and at least one quantity of food ingested.
Retrieving a level of physical and/or emotional activity from a user statement, a heart pulse monitoring system, a photoplethysmograph, an electrocardiograph or an electroencephalograph;
Predicting by at least one Recurrent Neural Network at least one glycemia at prediction time t+Δt from the glycemia data, the signals generated and the level of physical and/or emotional activity.
Retrieving at least one type of medication administered to the patient and at least one corresponding quantity of medication administered, the type of medication being chosen among Metformin, Sulfonylureas, Meglitinides, DPP-4 inhibitors, GLP-1 receptor agonists, SGLT2 inhibitors,
Predicting by at least one Recurrent Neural Network at least one glycemia at prediction time t+Δt from the glycemia data, the signals generated and at least one quantity of medication administered to the patient.

Completing the method with those specific steps open the use of the method to other populations. First, patients with diabetes under insulin therapy that also follow a treatment under one of several medications as recited above. Also, patients with type 2 diabetes under one of several medications as recited above that do not follow an insulin therapy. In this case, the quantity of insulin injected in the patient would of course set to zero. That does not raise any obstacle in successfully running the method.
Generating, by a plurality of Recurrent Neural Network arranged in parallel, a plurality of predicted glycemia corresponding to a plurality of prediction times, wherein at least one Recurrent Neural Network is of Long Short Term Memory type.

The method may feature a Root Mean Square Error (RMSE) of a predicted glycemia for a prediction time of 30 minutes compared to the temporally corresponding measured glycemia below 30 mg/dL, preferably below 20 mg/dL and even more preferably below 10 mg/dL, and a glycemia level and a Root Mean Square Error (RMSE) of the predicted glycemia for a prediction time of 90 minutes compared to the temporally corresponding measured glycemia is below 50 mg/dL, preferably below 40 mg/dL and even more preferably below 30 mg/dL.

The insulin blood concentration signal, the carbs rate of appearance signal, and optionally the free fatty acid blood concentration signal, may be generated through resolving at least one second order system of equations for each signal.

The method may be appropriately completed by the following steps, alone or in any of their technically possible combinations:
Retrieving amounts of rapid acting Uᵢ and long-acting U_{i,2} insulin injected in the patient;
Resolving the second order system of equations:

$$\{ \begin{matrix}{\dot{X}}_{2} = - {ω}_{i} ( {X}_{2} - {X}_{1} ) - {k}_{l} {X}_{2} ( t ) \\ {\dot{X}}_{1} = - {ω}_{i} ( {X}_{1} - {U}_{i} )\end{matrix}$$

Wherein
U₁ is the amount of rapid-acting insulin injected in the patient,
X₁ is the rapid-acting insulin concentration in a fictive intermediate compartment,
X₂ is the rapid-acting insulin blood concentration and
the parameter ωᵢ is the rapid-acting insulin transfer rate between compartments, and
the parameter kₗ represents irreversible degradation of insulin in the liver.
Resolving the second order system of equations

$$\{ \begin{matrix}{\dot{X}}_{2 , 2} = - {ω}_{i , 2} ( {X}_{2 , 2} - {X}_{1 , 2} ) - {k}_{l} {X}_{2 , 2} \\ {\dot{X}}_{1 , 2} = - {ω}_{i , 2} ( {X}_{1 , 2} - {U}_{i , 2} )\end{matrix}$$

Wherein
U_{i,2} is the amount of long-acting insulin injected in the patient,
X_{1,2} is the long-acting insulin concentration in a fictive intermediate compartment,
X_{2,2} is the long-acting insulin blood concentration and
the parameter ω_{i,2} is the long-acting insulin transfer rate between compartments, and
the parameter k₁ represents irreversible degradation of insulin in the liver.
Retrieving an amount of proteins ingested by the patient;
Adding a virtual amount of ingested glucose equal to 60% of the amount of protein ingested to form an effective amount Uᵣ of ingested glucose
Resolving the second order system of equations:

$$\{ \begin{matrix}{\dot{D}}_{2} = - {ω}_{r 2} ( {D}_{2} - {D}_{1} ) \\ {\dot{D}}_{1} = - {ω}_{r 1} ( {D}_{1} - {U}_{r} )\end{matrix}$$

Wherein
D₁ is the glucose concentration in a fictive intermediate compartment,
D₂ is the glucose blood concentration, and
the parameters ωᵣ₁ and ωᵣ₂ are glucose transfer rates between compartments.
Retrieving an amount U_{FFA} of free fatty acids ingested by the patient;
Resolving the second order system of equations:

$$\{ \begin{matrix}{\dot{F}}_{2} = - {ω}_{F F A} ( {F}_{2} - {F}_{1} ) \\ {\dot{F}}_{1} = - {ω}_{F F A} ( {F}_{1} - {U}_{F F A} )\end{matrix}$$

Wherein
F₁ is the free fatty acids concentration in a fictive intermediate compartment,
F₂ is the free fatty acids blood concentration and the parameter ω_{FFA} is the free fatty acids transfer rate between compartments.
Calibrating the pharmacokinetic pre-processing device, and determining the parameters involved in the above-mentioned second order systems of equations.

The calibration of the pharmacokinetic pre-processing device may be accomplished through a hybrid method combining a Nelder-Mead algorithm and a Particle Swarm Optimization method, including the steps of:
Creating an error function ƒ depending on the parameters involved in the second order systems of equations able to compare a predicted glycemia and a measured glycemia;
Generating a first set of p parameters corresponding to classical values, where p is the number of parameters involved in the second order systems of equations;
Logging the set of p parameters in the pharmacokinetic pre-processing device;
Running the pharmacokinetic pre-processing device using historical user data to predict at least one glycemia;
Calculating the value of the error function f depending on p parameters involved in the second order systems of equations;
Comparing the value of the error function f to a predetermined threshold;
According to the result of the comparison terminating the method and keeping the first set of p parameters as optimized parameters or else performing an optimization process loop that includes the steps of:
   Generating randomly a set of V vectors Xi of dimension p representing V sets of p parameters;
   Generating for each of the V vectors Xi a trajectory Ti of successive vectors X_{i,n} each corresponding to a time step n, the transit from one vector of time step n to the following time step n+1 including the steps of:
      Identifying (E1) X_{i best} corresponding to the minimum value of ƒ calculated among the vectors of the trajectory Ti corresponding to a maximum time step n;
      Identifying (E2) X_{j≠i best} corresponding to the minimum value of ƒ calculated among the vectors of all trajectories but Ti and corresponding to a maximum time step n;
      Determining (E3) a vector Y_{i,n+1} with the Particle Swarm Optimization method formula:

Y_{i,n+1}=c₁*X_{i,n}+c₂*[X_{i,best}−X_{i,n}]+c₃*[X_{j≠i best}−X_{i,n}]
wherein c₁, c₂, c₃ are parameters so that c₁=1.8
0≤c₂≤1.62 and 0≤c₃≤1.62;
   Applying (E4) a Nelder-Mead algorithm step to each Y_{i,n+1}, in order to produce a vector X_{i,n+1} local minimum of the function ƒ using a simplex of (p+1) vectors ₘZ_{i,n+1}, with m∈ [symbol] 1, p+1 [symbol] , each vector of dimension p defined by ₘZ_{i,n+1}^{k}=Y_{i,n+1}^{k}(1+m*δ) with k∈ [symbol] 1, p [symbol] and δ=0.05, wherein Y_{i,n+1}^{k} is the k-th coordinate of the vector Y_{i,n+1} and ₘZ_{i,n+1}^{k} is the k-th coordinate of the vector ₘZ_{i,n+1};
   Comparing (E5) the value of ƒ(X_{i,n+i}) and a predetermined threshold;
   Repeating steps (E1) to (E5) until a value of ƒ(X_{i,n+1}) falls below the predetermined threshold or else terminating the method, the corresponding X_{i,n+1} being the set of optimized parameters.

The method may also include a step of offering to the user to realize short and simple experiences in order to retrieve the corresponding data.

The invention also relates to a method for recommending a food quantity to ingest including the steps of:
Retrieving a target glycemia interval to be reached at a certain future time t+Δt;
Retrieving a data set of glycemia previously measured on a patient;
Choosing an initial and virtual quantity of food to ingest by the patient;
Applying the glycemia prediction method as described above to produce at least one predicted glycemia at time t+Δt;
Recursively determining a data set URf(t) of dated food quantity to ingest by the patient that correspond to a predicted glycemia inside the target glycemia interval at time t+Δt,
Displaying the corresponding recommendation to the patient.

This method for recommending a food quantity to ingest may in particular be used for non-diabetic people, such as athletes or people interested in achieving physical performances. An insulin peak associated with a postprandial blood glucose or a low blood sugar level could cause a drop in performance, loss of concentration . . . Predict the blood glucose will help anticipate these events and act consistently to prevent them (ingestion of optimal amount of carbs, adjustment of nutrients, etc. . . . ).

The method may also further include the steps of
Retrieving a constraint possibly containing:
   a maximum duration of time above a glycemia level not to exceed;
   a minimum glycemia level below which not to fall;
   a wish to perform a physical exercise;
Choosing an initial and virtual level of physical intensity;
Recursively determining the data set URf(t) and a data set URa(t) of dated level of physical exercise intensity to perform by the patient so that:
   predictions of glycemia by the prediction system do not exceed the glycemia level longer than the maximum duration of time
   predictions of glycemia by the prediction system all stand above the minimum glycemia level,
   URa(t) is compatible with the wish to perform a physical exercise
Displaying the corresponding recommendation to the patient.

The invention also relates to a method for recommending an insulin quantity to inject including the steps of:
Retrieving a target glycemia interval to be reached at a certain future time t+Δt;
Retrieving a data set of glycemia previously measured on a patient;
Choosing an initial and virtual quantity of insulin to inject in the patient;
Applying the glycemia prediction method as described above to produce at least one predicted glycemia at time t+Δt;
Recursively determining a data set URi(t) of dated amount of insulin to inject in the patient that correspond to a predicted glycemia inside the target glycemia interval at time t+Δt,
Displaying a message to the patient mentioning the determined amount of insulin to inject.
   This method for recommending an insulin quantity to inject may in particular be used for patients with type 1 or type 2 diabetes that are under insulin treatment.

The method may also further include the steps of
Retrieving a constraint possibly containing:
   a maximum insulin quantity to inject;
   a maximum duration of time above a glycemia level not to exceed;
   a minimum glycemia level below which not to fall;
   a wish to perform a physical exercise;
Choosing an initial and virtual level of physical intensity;
Recursively determining the data set URi(t) and a data set URa(t) of dated level of physical exercise intensity to perform by the subject so that:
   predictions of glycemia by the prediction system do not exceed the glycemia level longer than the maximum duration of time
   predictions of glycemia by the prediction system all stand above the minimum glycemia level,
   URi(t) is compatible with the maximum quantity of insulin to inject
   URa(t) is compatible with the wish to perform a physical exercise
Displaying the corresponding recommendation to the subject.

The invention also relates to insulin for use in the treatment of diabetes characterised in that the dosage regimen is determined by the method mentioned above

A pharmaceutical composition for treating type 1 diabetes is proposed comprising comprising insulin characterised in that the dosage regimen is determined by the method mentioned above.

The invention also relates to a glycemia prediction device comprising:
a glycemia monitoring system able to measure the glycemia of the patient and transfer data of dated glycemia measures of the patient,
a first device able to retrieve and transfer data of dated insulin quantity injected in the patient,
a second device able to retrieve and transfer data of dated food type and quantity ingested by the patient,
an optional third device able to retrieve and transfer data of dated physical and/or emotional activity of the patient
a pharmacokinetic pre-processing device linked to the glycemia monitoring system, to the first, second and optional third devices, able to generate from the data sets of dated measured glycemia, dated insulin quantity injected and dated food type and quantity ingested, an insulin blood concentration signal, a carbs rate of appearance signal, and optionally a free fatty acid blood concentration signal.
a prediction system comprising at least one Long Short Term Memory neural network and linked to the pharmacokinetic pre-processing device, able to produce from the data sets of dated measured glycemia, optionally dated physical and/or emotional activity and the signals generated at least one predicted glycemia.

The invention also relates to a glycemia recommendation device comprising:
a glycemia prediction device as mentioned above,
a recursive data generator linked to the output, to the first, second and optional third devices of the glycemia prediction device and able to create and modify according to the output of the glycemia prediction device a virtual data set of dated discrete insulin quantity to inject into the patient, a virtual data set of dated discrete food quantity to ingest by the patient, and an optional virtual data set URa(t) of dated level of physical exercise intensity to perform by the patient
a first entry of the data generator to retrieve a target glycemia interval to be reached at a certain horizon,
a second optional entry of the data generator to retrieve a constraint possibly containing
   a maximum insulin quantity to inject;
   a maximum duration of time above a glycemia level not to exceed;
   a minimum glycemia level below which not to fall;
   a wish to perform a physical exercise;
a displayer to present the recommendation to the patient

### BRIEF DESCRIPTION OF THE FIGURES

Other features, objects and advantages of the present invention will become apparent upon reading the detailed description which follows, with reference to the appended figures, given as non-limiting examples and wherein:

FIG. 1 is a block diagram illustrating an example of the glycemia prediction method.

FIG. 2 is a block diagram illustrating an example of the optimization of the pharmacokinetic model.

FIG. 3 is a block diagram illustrating an example of the glycemia recommendation method.

### DETAILED DESCRIPTION OF THE EMBODIMENTS

The following modes, given by way of example only, are described in order to provide a more precise understanding of the subject matter of a preferred embodiment or embodiments.

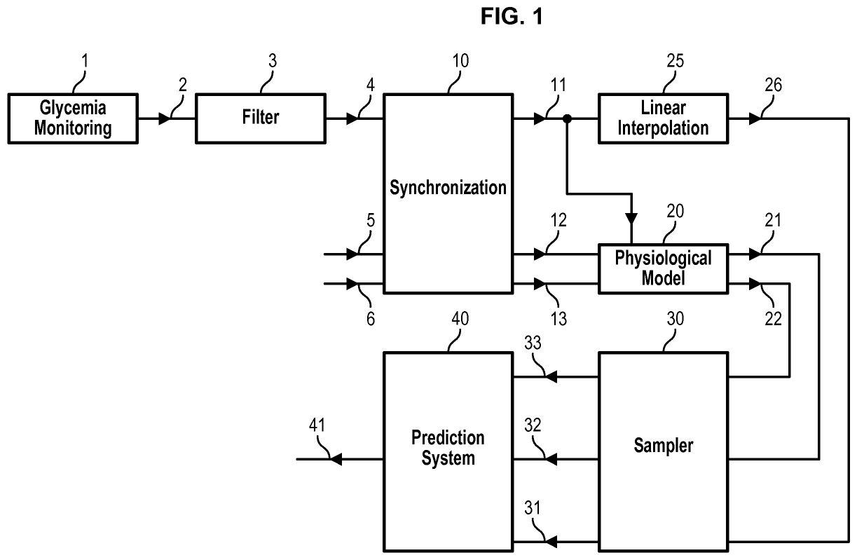

An example of the glycemia prediction method is described on FIG. 1.

A data set 2 of dated glycemia levels is retrieved from a device 1 to monitor the glycemia of the user.

Such a device may be a continuous glucose monitoring device.

This kind of devices usually requires to be filtered before any data processing.

The data set 2 is transformed by a smoothing or de-noising Filter 3 into a filtered dated discrete measured glycemia data set 4.

Yet, the last measure of glycemia cannot be de-noised as efficiently as the rest of the data set. That is why this measure is weighted of less importance compared to the older measures, the difference of weight being used further in the process.

As such, the glycemia prediction method does not require a glycemia measurement step.

The glycemia prediction method requires dated glycemia levels previously measured as an input, and preferably filtered dated glycemia levels.

The glycemia prediction method requires a retrieving step of a data set 2 of dated glycemia levels. The retrieved data set may be already de-noised or a filtering step to de-noise the data set may be part of the glycemia prediction method.

The dated quantity of insulin 5 injected into the user is retrieved either by declaration of the user or transmitted by an automatic management device such as an insulin pump, or a connected insulin pen.

In the case of non-diabetic people or patients with diabetes under other medication than insulin, the dated quantity of insulin 5 injected should be set to zero. That does not raise any obstacle to successfully run the method whatsoever.

The dated quantity of insulin 5 injected into the user may also be divided into two quantities of insulin, a quantity of rapid-acting insulin and a quantity of long-acting insulin.

The dated quantity of food 6 ingested by the user is retrieved either by declaration of the user or transmitted by a device able to estimate food intakes for example through a declaration of the user, possibly using food composition tables or a predetermined list of meals, or a photographic snapshot of the meal which is then digitally processed.

In particular, the estimation of food intakes includes an estimation of the amount of ingested glucose, but may also include other types of food such as lipids, free fatty acids, proteins and fibers.

Other data, such as measures of heart beating rate or brain activity level may also be retrieved in order to take into account the physical and emotional activities of the user.

Those data may be retrieved from an activity tracker such as a heart pulse monitoring system, a photoplethysmograph, an electrocardiograph or an electroencephalograph.

Those data may also be directly filled by the user.

Those data may further be transformed in qualitative data corresponding to a level of physical and/or emotional intensity.

Signals 4, 5 and 6, and possibly the qualitative data of physical and/or emotional intensity are synchronized through a synchronization device 10 to temporally set the different measures one against the other.

Synchronized glycemia signal 11, injected insulin signal 12 and food intake signal 13 are generated by said synchronization device 10.

Further, the synchronized glycemia signal 11 is sent through a linear interpolation device 25 in order to generate a continuous glycemia signal 26.

Signals 11, 12 and 13 are sent into a Pharmacokinetic pre-processing device 20 in order to produce an estimation of the blood insulin concentration 21 and an estimation of the blood carbs rate of appearance 22.

In the case where a qualitative data of physical and emotional intensity is available, the Pharmacokinetic pre-processing device 20 may take it into account to produce the estimations.

The Pharmacokinetic pre-processing device holds in a memory support a knowledge-driven model of the glucose-insulin system.

The model belongs to compartmental models that are traditionally used in pharmacokinetics and physiology to describe both transfer and degradation mechanisms of specific substances in the human body among multiples compartments that can be fictive or related to body organs. Compartmental modelling may allow predictions of the blood concentration of those specific substances.

As the Dalla Man model first requires to evaluate many parameters for the patient, and thus complex clinical trials and secondly does not take into account the physical activity of the patient, a simpler model is used in the Pharmacokinetic pre-processing device:

Both glucose and insulin appearance rate are modelled through second order system of equations.

The insulin rate is modelled through the following system of the following type E_{I}:

$$\{ \begin{matrix}{\dot{X}}_{2} = - {ω}_{i} ( {X}_{2} - {X}_{1} ) - {k}_{l} {X}_{2} ( t ) \\ {\dot{X}}_{1} = - {ω}_{i} ( {X}_{1} - {U}_{i} )\end{matrix}$$

Wherein
Uᵢ is the amount of insulin injected in the patient,
X₁ is the insulin concentration in a fictive intermediate compartment,
X₂ is the insulin blood concentration and
the parameter ωᵢ is the insulin transfer rate between compartments, and
the parameter kₗ represents irreversible degradation of insulin in the liver.
Rapid-acting and long-acting insulins may be both taken into account in the model using two systems of equations, each corresponding to rapid-acting or long-acting insulin, each of the type E_{I}, but featuring different parameters ωᵢ.

The glucose rate is modelled through the following system of the following type E_{G}

$$\{ \begin{matrix}{\dot{D}}_{2} = - {ω}_{r 2} ( {D}_{2} - {D}_{1} ) \\ {\dot{D}}_{1} = - {ω}_{r 1} ( {D}_{1} - {U}_{r 1} )\end{matrix}$$

Wherein
Uᵣ₁ is the amount of ingested glucose,
D₁ is the glucose concentration in a fictive intermediate compartment,
D₂ is the glucose blood concentration, and
the parameters ωᵣ₁ and ωᵣ₂ are glucose transfer rates between compartments.

The presence of fibers in the meal over a certain threshold would tend to slow down the kinetics of glucose absorption. To model the impact of glucose ingested along with fibers, a second order system of equations of type E_{G} may be resolved in order to establish carbs rate of appearance signal featuring lower parameters ωᵣ₁ and ωᵣ₂ than in the case of glucose ingested with no fibers.

The presence of proteins in a meal lead to a further apparition of blood glucose. 60% of the amount of absorbed proteins may be considered as further converted into glucose. To model the impact of protein ingested, the second order system of equations corresponding to glucose ingested with may be resolved using a virtual amount of ingested glucose equal to 60% of the amount of protein ingested, replacing the amount of ingested glucose by an effective amount of ingested glucose Uᵣ.

The presence of free fatty acids in a meal lead to a further apparition of free fatty acids blood concentration that impact the glycemia. To model this apparition, the second order system of equations may be resolved

$$\{ \begin{matrix}{\dot{F}}_{2} = - {ω}_{F F A} ( {F}_{2} - {F}_{1} ) \\ {\dot{F}}_{1} = - {ω}_{F F A} ( {F}_{1} - {U}_{F F A} )\end{matrix}$$

Wherein
F₁ is the free fatty acids concentration in a fictive intermediate compartment,
F₂ is the free fatty acids blood concentration and
the parameter ω_{FFA} is the free fatty acids transfer rate between compartments.

The knowledge-driven model of the glucose-insulin system may also be run to further predict glycemia.

For instance, the equation regulating glycemia G may be given by:

Ġ=EGP₀+Rₐ(t)−U_{id}(t)−Uᵢᵢ(t)−E(t)
wherein:
EGP₀ is a parameter corresponding to glucose production in absence of insulin,
Rₐ(t)=k_{gr}D₂(t), and k_{gr} is a parameter corresponding to the action of the glucose input on the glycemia,

$${U}_{i d} ( t ) = \{ \begin{matrix}{p}_{2} G ( t ) {e}^{{S}_{I} {X}_{2} ( t )} & if {F}_{2} ( t ) = 0 \\ {p}_{2} G ( t ) {e}^{{S}_{I , FFA} \frac{{X}_{2} ( t )}{{F}_{2} ( t )}} & if {F}_{2} ( t ) > 0\end{matrix}$$

p₂ is a parameter corresponding to the rate of insulin-dependent glucose utilization
Sₗ is a parameter corresponding to the action of the insulin input on the glycaemia
S_{l,FFA} is a parameter corresponding to the action of the insulin input on the glycaemia in the presence of free fatty acids.
X₂ is the insulin blood concentration, sum of rapid-acting and long-acting insulin blood concentrations.
Uᵢᵢ(t)=p₁G(t) and p₁ is a parameter corresponding to the rate of insulin-independent glucose utilization

$$E ( t ) = \{ \begin{matrix}{k}_{ge} \cdot [ G ( t ) - {k}_{gt} ] & if G ( t ) > {k}_{gt} \\ 0 & if G ( t ) \leq {k}_{gt}\end{matrix}$$

k_{ge} is a parameter corresponding to the rate of glucose renal excretion
k_{gt} is a parameter corresponding to the glycemia value from which the kidney has an action on blood sugar level.

The different parameters appearing in the previous equations are requested to run the model.

The requested parameters of the model may be classical values, such as statistical mean values extracted from general studies, or they may be calibrated on the user.

In this last situation, a calibration process of the Pharmacokinetic pre-processing device is required.

This calibration may be performed regularly in order to ensure a relevant estimation of blood insulin concentration and blood carbs rate of appearance.

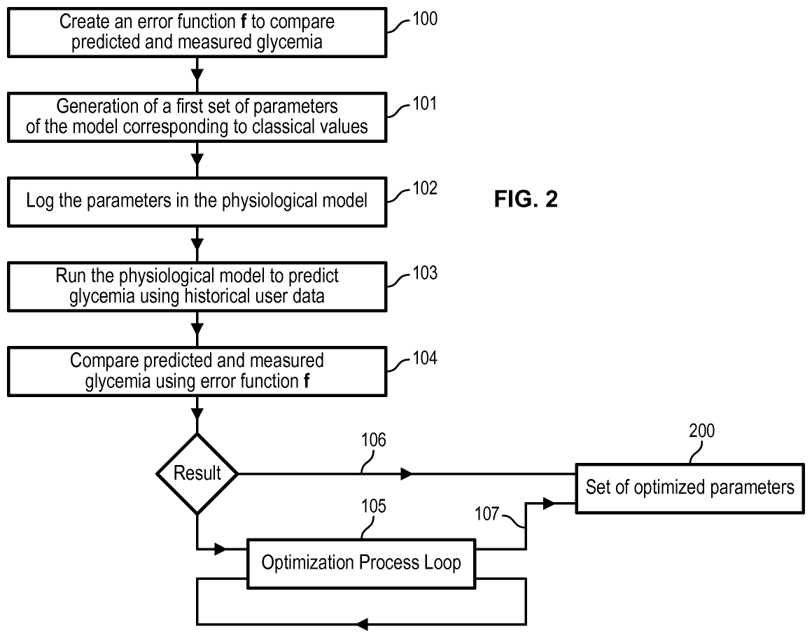

The FIG. 2 illustrates an example of a calibration process.
The step 100 consists in creating an error function ƒ depending on the parameters involved in the second order systems of equations used in the knowledge-driven model of the glucose-insulin system, the function being able to compare a predicted glycemia and a measured glycemia;
The step 101 consists in generating a first set of p parameters corresponding to classical values, where p is the number of parameters involved in the second order systems of equations;
The step 102 consists in logging those parameters in the memory of the Pharmacokinetic pre-processing device.
The step 103 consists in running the knowledge-driven model of the glucose-insulin system in order to predict glycemia. The parameters in the equation regulating glycemia G are classical values, such as statistical mean values extracted from general studies. This step involves an historical data set retrieved on the patient that includes measured and timestamped glycemia, dated quantity of insulin injected into the user and dated quantity of food ingested by the user.
The step 104 consists in comparing the predicted glycemia and the corresponding measured glycemia by calculating the value of the error function ƒ. This step implies that the historic data set is important enough to ensure that sufficient measured glycemia corresponding to the predicted data are part of the data set.

In the case where the value of the error function is superior to a predetermined threshold an optimization process loop 105 is performed to modify the parameters.

At least one new set of parameters is generated and modified to be brought closer to the corresponding measured glycemia by minimizing the value of the error function ƒ.

Once the value of the error function ƒ is below a predetermined threshold, current parameters are considered in step 200 as optimized parameters and optimization process is over.

The optimization process loop 105 may be performed thanks to different algorithms.

In particular, a hybrid method combining Nelder-Mead algorithm with Particle Swarm Optimization (PSO) method may be used in order to optimize the values of the requested parameters of the model according to the measures carried out on the subject and calibrate the Pharmacokinetic pre-processing device.

The Nelder-Mead method generalizes the triangle notion in a finite dimensional space; in order to find a local minimum of a function ƒ of p variables.

The steps described below are implemented:
1. Create a “p+1 simplex”, a set of p+1 vectors w₁, w₂, . . . , wₚ, wₚ₊₁ each of dimension p.
2. Calculate the values of ƒ for each vector of the simplex.
3. Sort the p+1 values by increasing order. Index again the simplex to obtain:

ƒ(w₁)<ƒ(w₂) . . . <ƒ(wₚ)<ƒ(wₚ₊₁)
4. Calculate the centroid of the p smallest values:

$${w}_{0} = \frac{1}{p} {Σ}_{h = 1}^{p} {w}_{h}$$

5. Calculate the reflection wᵣ. of wₚ₊₁ across the point w₀:

wᵣ=w₀+a*(w₀−wₚ₊₁), with a=1.
6.
   a. If ƒ(wₚ)>ƒ(wᵣ)>ƒ(w₁), replace wₚ₊₁ by wᵣ and start again from step 3.
   b. If ƒ(w₁)>ƒ(wᵣ), calculate an expanded point wₑ.

wₑ=w₀+γ*(wᵣ−w₀), with γ=2.
i. If ƒ(wₑ)<ƒ(wᵣ), replace wₚ₊₁ by wₑ and start again from step 3.
ii. Else replace wₚ₊₁ by wᵣ and start again from step 3.
c. If ƒ(wₚ₊₁)>ƒ(wᵣ)>ƒ(wₚ) calculate a contracted point w_{cout}

w_{cout}=w₀+ρ*(wᵣ−w₀), with ρ=½.
i. If ƒ(w_{cout}<ƒ(wᵣ), replace wₚ₊₁ by w_{cout}, and start again from step 3.
ii. Else go to step 7
d. If ƒ(wᵣ)≥ƒ(wₚ₊₁) calculate a contracted point w_{cin}.:

w_{cin}=w₀+ρ*(wₚ₊₁−w₀)
i. If ƒ(w_{cin})<ƒ(wₚ₊₁), replace wₚ₊₁ by w_{cin}, and start again from step 3.
ii. Else go to step 7
7. Shrink all the vectors of the simplex but w₁

∀h∈[2,p+1]: wₕ=w₁+σ*(wₕ−w₁), with σ=½
and start again from step 3.

To stop the iteration of the algorithm, a threshold for ƒ(w₁) or a specified number of iterations for a specific lowering of ƒ(w₁) is needed.

The higher the value of the threshold, the longer the process and the more accurate the position of the local minimum.

The value of the threshold may then be tuned according to the constraints of time and accuracy.

This algorithm is easy to implement and efficient to find a local minimum of the function ƒ. However, it is not convenient for finding the global minimum in the case where ƒ features multiple local minima.

Applying this algorithm to optimize the values of the requested parameters of the model according to the measures carried out on the subject and calibrate the Pharmacokinetic pre-processing device, enables to determine quickly a set of parameters that minimizes locally the error function.

Yet, it is impossible to be sure that the minimum is a global minimum.

The PSO is a second optimization method, inspired by flocks of birds and schools of fish.

The PSO is an algorithm able to explore the entire problem space.

A swarm of vectors is needed to begin the algorithm.

At each iteration, particles move in the problem space according to the previous movement and two formulas: the first is based on a cognitive behavior and the second on a social behavior.

The steps described below are implemented to determine a global minimum of a function ƒ of p variables:
1. Generating randomly a set of V vectors Xi of dimension p;
2. Generating for each of the V vectors Xi a trajectory Ti of successive vectors X_{i,n} each corresponding to a time step n, the transit from one vector of time step n to the following time step n+1 including the steps of:
   Identifying (R1) X_{i best} corresponding to the minimum value of ƒ calculated among the vectors of the trajectory Ti corresponding to a maximum time step n;
   Identifying (R2) X_{j≠i best} corresponding to the minimum value of ƒ calculated among the vectors of all trajectories but Ti and corresponding to a maximum time step n;
   Determining (R3) a vector X_{i,n+1} with the formula:

X_{i,n+1}=c₁*X_{i,n}+c₂*[X_{i,best}−X_{i,n}]+c₃*[X_{j≠i best}−X_{i,n}]
To prevent a routine, random variables can be included in this formula at each iteration.
   wherein c₁, c₂, c₃ are parameters chosen so that c₁=1.8 0≤c₂≤1.62 and 0≤c₃≤1.62
Comparing (R4) the value of ƒ(X_{i,n+1}) and a predetermined threshold;
Repeating steps (R1) to (R4) until a value of ƒ(X_{i,n+1}) falls below the predetermined threshold.

The cognitive behavior guides the particle movement towards its best previous position, it is characterized in the algorithm with the parameter c₂.

The social behavior guides the particle movement towards the best previous position of its neighborhood, it is characterized in the algorithm with the parameter c₃.

The movement of the particles varies according to the ratio between c₂. and c₃.:

When c₂.=c₃, social and cognitive behaviors are of equal influence and particles cannot be stuck in a local minimum. The minimum of the function ƒ of p variables determined by the algorithm is sure to be the global minimum, although the determination is long.

When c₂.<c₃, social behavior is of greater influence and particles can be stuck in a local minimum. The determination is shorter but the minimum of the function ƒ of p variables determined by the algorithm is not sure to be the global minimum.

Downsides of the Nelder-Mead and PSO methods can be reduced by combining them: applying the Nelder Mead algorithm after each particle move of the PSO algorithm ensures to determine the greatest local minimum around each particle position. The random part of the calculation time is lowered.

The steps described below are implemented to determine a global minimum of a function ƒ of p variables:
1. Generating randomly a set of V vectors Xi of dimension p representing V sets of p parameters;
2. Generating for each of the V vectors Xi a trajectory Ti of successive vectors X_{i,n} each corresponding to a time step n, the transit from one vector of time step n to the following time step n+1 including the steps of:
   Identifying (E1) X_{i best} corresponding to the minimum value of ƒ calculated among the vectors of the trajectory Ti corresponding to a maximum time step n;
   Identifying (E2) X_{j≠i best} corresponding to the minimum value of ƒ calculated among the vectors of all trajectories but Ti and corresponding to a maximum time step n;
   Determining (E3) a vector Y_{i,n+1} with the Particle Swarm Optimization method formula:

Y_{i,n+1}=c₁*X_{i,n}+c₂*[X_{i,best}−X_{i,n}]+c₃*[X_{j≠i best}−X_{i,n}]
wherein c₁, c₂, c₃ are parameters so that c₁=1.8
0≤c₂≤1.62 and 0≤c₃≤1.62;
Applying (E4) a Nelder-Mead algorithm step to each Y_{i,n+1}, in order to produce a vector X_{i,n+1} local minimum of the function ƒ using a simplex of (p+1) vectors ₘZ_{i,n+1}, with m∈ [symbol] 1, p+1 [symbol] , each vector of dimension p defined by ₘZ_{i,n+1}=Y_{i,n+1}^{k}(1+m*δ) with k∈ [symbol] 1, p [symbol] and δ=0.05, wherein Yₙ₊₁^{k} is the k-th coordinate of the vector Y_{i,n+1} and ₘZ_{i,n+1}^{k} is the k-th coordinate of the vector ₘZ_{i,n+1};
Comparing (E5) the value of ƒ(X_{i,n+1}) and a predetermined threshold;
Repeating steps (E1) to (E5) until a value of ƒ(X_{i,n+1}) falls below the predetermined threshold or else terminating (107) the method terminating the method, the corresponding X_{i,n+1} being the set of optimized parameters.

The Pharmacokinetic pre-processing device produces signals 21 and 22 that are continuous.

Signals 21, 22 and 26 are sampled through a sampler 30 using the same sampling rate for the different incoming signals.

Sampled glycemia signal 31, blood insulin concentration 32 and carbs rate of appearance 33 are generated.

Those signals are sent into a prediction system 40 comprising at least one recurrent neural network or a combination of a Support Vector Machine (SVM) and a recurrent neural network.

Other signals, such as a qualitative data corresponding to a level of physical and/or emotional intensity may also be sent into the prediction system 40.

Neural networks feature an input layer where the input is received, an output layer and optionally between them one or more hidden layer.

The output of each hidden layer is used as input to the next layer in the network, that is the next hidden layer or the output layer. Each layer of the network generates at each time step an output from its current received input in accordance with current values of a respective set of parameters.

Recurrent neural networks can use some or all of the internal state of the network from a previous time step in computing an output at a current time step.

At least one predicted glycemia g(t+Δt) 41 for one prediction horizon Δt is generated at the output of the prediction system.

A particular embodiment can be realized using a prediction system comprising a plurality of different neural networks arranged in parallel.

Each neural network corresponds to a specific prediction horizon and works independently from the others.

A particular embodiment can be realized using recurrent neural networks and more particularly neural networks of the LSTM type (Long-Short Term Memory). The LSTM neural networks are presented in the application US2017/0228642.

The neural network can offer reliable predictions only after a training period.

During this period, the predictions produced by the neural network fora specific prediction time, are compared to the corresponding real measured value of glycemia.

This comparison data is collected all along the training period so that the neural network learns how to improve its predictions.

The training period is directly linked to the recorded data set of experiences of the user relevant regarding the prediction of his glycemia.

In order to speed up the training period, the system may offer to the user to realize short and simple experiences in order to retrieve the corresponding data and complete a lack in the current recorded data set.

When the absolute value of the difference between real and predicted glycemia falls below a certain threshold, the training period terminates.

The neural network may then be used with an error level corresponding to the said threshold.

When the prediction system is run, the glycemia predictions of the neural network still remain compared to the measured levels of glycemia.

Whenever the absolute value of the difference between measured and predicted glycemia exceeds the threshold, a new training period is launched.

In order to minimize the length of a training period, it is important that:
the input signals sent in the neural network correspond to physical quantities the most relevant possible to predict the glycemia level,
the number of different input signals sent in the neural network is the lowest possible,
the number of layers inside the neural network is the lowest possible.

Also, the lower the prediction horizon and the shorter the training period.

Traditional RNNs with a reduced input of physical quantities or a reduced number of layers are less efficient to learn dependencies and to learn how to connect the information

In the case of glycemia prediction, it is difficult to reduce the length of a training period and maintain or reduce the error level.

As an example of traditional RNNS, the Elman Recurrent Neural Network (ERNN), also known as Simple or Vanillan RNN, is usually considered to be the most basic version of RNN. The layers in ERNN can be divided in input layers, hidden layers and the output layers. While input and output layers are characterized by feedforward connections, the hidden layers contain recurrent ones.

The input of the ERNN is a time series {xₜ₋ₙ₊₁, xₜ₋ₙ₊₁, . . . , xₜ} or {x[t−n+1], x[t−n+2], . . . x[t]} of n ordered vectors. The time series has length T and it can contain real values, discrete values, one-hot vectors, and so on . . . . At time step u, the input layer process the component x[u] of the time series. In the input layer, each component x[u] is summed with a bias vector bᵢ and then is multiplied with an input weight matrix Wᵢ^{h}. The internal state of the network h[u−1] from the previous time step is first summed with a bias vector bₕ and then multiplied by the weight matrix Wₕ^{h} of the recurrent connections. Wₕ^{h} is often called Context matrix. The transformed current input and past network state are then combined and processed by the neurons in the hidden layers, which apply a non-linear transformation ƒ usually implemented by a sigmoid or a hyperbolic tangent. The equation defining the hidden state h[u] is the following:

h[u]=ƒ(Wᵢ^{h}(x[u]+bᵢ)+Wₕ^{h}(h[u−1]+bₕ))

The hidden state h[u] conveys the content of the memory of the network at time step u. It is typically initialized with a vector of zeros and it depends on past inputs and network states.

All the weight matrices and biases can be trained through gradient descent, according to a BPTT (BackPropagation Through Time) procedure.

During the BPTT, a loss function is used to calculate the difference between the network output and its expected output, after a case propagates through the network.

An optimization algorithm is used to propagate forward through the network, layer by layer, an input vector until it reaches the output layer. The output is compared to the desired output using the loss function. The resulting error value is calculated for each of the neurons in the output layer, and propagated back through the network, until each neuron has an associated error value that reflects its contribution to the original output.

Backpropagation uses these error values to calculate the gradient of the loss function. This gradient is then fed to an optimization algorithm which updates weights, in attempt to minimize the loss function.

The “vanishing gradient problem” is a major obstacle to recurrent net performance like Elman nets.

Recurrent nets try to establish connections between a final output and events many time steps before. But the task is difficult because the information flowing through neural nets passes through many stages of multiplication. In particular, after n iterations, hidden state h[u] comprises information from component x[u−n] that has been multiplied n times by the weight matrix Wₕ^{h} and processed n times by the non-linear transformation. Because any quantity multiplied frequently by an amount less than one can become too small for computers to work with, applying a sigmoid function over and over again can make a gradient vanish.

And if the gradient vanishes, it is impossible to adjust the weights and the network cease to learn.

Long Short Term Memory (LSTM) networks solve this problem.

LSTM is a specific type of Recurrent Neural Network.

As opposed to the input of regression algorithms, such as SVM, consisting of single vectors, the input to the LSTM network is a time series {xₜ₋ₙ₊₁, xₜ₋ₙ₊₂, . . . , xₜ} of n ordered vectors.

These vectors are timestamped, with a specified time span (for instance 15 minutes).

Each vector includes data such as glycemia concentration, insulin treatment data, quantity and type of food consumption, physical activity, etc. . . . .

As a Recurrent neural network (RNN) LSTM is made up of repeating feed-forward neural networks connected in series.

The process may be considered as repeating the same loop of operations contained in a cell.

At the i^{th} iteration, the operations of the cell are performed on the i^{th} vector xₜ₋ₙ₊ᵢ and involve the intermediate result hₜ₋ₙ₊₁ of the previous iteration and a context vector cₜ₋ₙ₊ᵢ₋₁ produced in the previous iteration.

The process may be considered as performing the operations contained in a series of identical cells connected.

The two considerations are equivalent.

According to the consideration of a series of identical cells connected, the LSTM network includes as many cells as vectors in the input sequence, and each vector in the sequence is chronologically fed to exactly one cell.

The i^{th} vector xₜ₋ₙ₊ᵢ in the sequence is the input of the i^{th} cell.

This difference of weight, generated during the filtering step in the data set of glycemia is used here in the process to take into account the last measure of glycemia that cannot be de-noised as efficiently as the rest of the data set.

The cells are connected in series according to the chronological order and passing information from one cell to the next in the series.

The i^{th} cell of the LSTM delivers an intermediate output hₜ₋ₙ₊ᵢ. The output of the last cell corresponds to the predicted glycemia.

The i^{th} cell receives a context vector cₜ₋ₙ₊ᵢ₋₁ and delivers a context vector cₜ₋ₙ₊ᵢ containing relevant information retrieved from previous cells, or in an equivalent way retrieved from previous iterations.

Thanks to four neural network layers contained in each cell and interacting in a very specific way, it is decided what information of the upcoming context vector should be forgotten, updated and outputted to the next one.

More specifically, a LSTM cell is composed of 5 different nonlinear components, interacting with each other in a particular way. The internal state of a cell is modified by the LSTM only through linear interactions. This permits information to backpropagate smoothly across time, with a consequent enhancement of the memory capacity of the cell. LSTM protects and controls the information in the cell through three gates, which are implemented by a sigmoid and a pointwise multiplication. To control the behavior of each gate, a set of parameters are trained with gradient descent, in order to solve a target task.

Each gate in the cell has a specific and unique functionality.

The transformation, for example in the i^{th} cell, follows three steps:
Decide what information is to be suppressed in the content of the received context vector cₜ₋ₙ₊ᵢ₋₁, thanks to a “forget gate” layer involving the intermediate output hₜ₋ₙ₊ᵢ₋₁. of the previous cell and the input vector xₜ₋ₙ₊ᵢ.
Decide what new information is to be to stored in the context vector cₜ₋ₙ₊ᵢ, an “input gate” layer decides which values are to be updated, and another layer creates a vector of new candidates values that could be added to the context vector. The results of the two layers are combined to create the context vector cₜ₋ₙ₊ᵢ. An “update gate” operates on the previous state cₜ₋ₙ₊ᵢ₋₁, after having been modified by the forget gate, and it decides how much the new state cₜ₋ₙ₊ᵢ should be updated with a new candidate \tilde{c}ₜ₋ₙ₊₁.

The equations defining the context vector cₜ₋ₙ₊ᵢ, are the following:

Forget Gate: σ_{ƒ}[t]=σ(W_{ƒ}xₜ₋ₙ₊ᵢ+R_{ƒ}hₜ₋ₙ₊ᵢ₋₁+b_{ƒ})

Candidate State: \tilde{c}ₜ₋ₙ₊ᵢ=g₁(Wₕxₜ₋ₙ₊ᵢ+Rₕhₜ₋ₙ₊ᵢ₋₁bₕ)

Update Gate: σ_{g}[t]=σ(W_{g}xₜ₋ₙ₊ᵢ+R_{g}hₜ₋ₙ₊ᵢ₋₁b_{g})

Cell State: cₜ₋ₙ₊ᵢ=σ_{g}[t]⊙\tilde{c}ₜ₋ₙ₊ᵢ+σ_{f}[t]⊙cₜ₋ₙ₊ᵢ₋₁
Each gate depends on the current external input xₜ₋ₙ₊ᵢ and the previous cells output hₜ₋ₙ₊ᵢ₋₁. W_{f}, Wₕ, W_{g}, and Wₒ are rectangular weight matrices, that are applied to the input of the LSTM cell. R_{f}, Rₕ, R_{g}, and Rₒ are square matrices that define the weights of the recurrent connections, while b_{f}, bₕ, b_{g}, and bₒ are bias vectors. The function σ(⋅) is a sigmoid, while g₁(⋅) and g₂(⋅) are pointwise non-linear activation functions, usually implemented as hyperbolic tangents that squash the values in [−1, 1]. Finally, ⊙ is the entrywise multiplication between two vectors (Hadamard product).
Decide the intermediate output hₜ₋ₙ₊₁ based on the context vector cₜ₋ₙ₊ᵢ. and thanks to a last layer: to produce the output hₜ₋ₙ₊ᵢ, first the cell filters its current state with a nonlinearity g2(⋅). Then, an output gate selects the part of the state to be returned as output.

The system may possibly be adapted so that σ_{f}=1 and σ_{g}=0, the current state of a cell is transferred to the next time interval exactly as it is, that is cₜ₋ₙ₊ᵢ=cₜ₋ₙ₊ᵢ₋₁

It is therefore possible that after n iterations, h[u] comprises unmodified information of component x[u−n] thanks to the context vector cₜ₋ₙ₊ᵢ.

That is why, in LSTM, the issue of vanishing gradient does not occur, due to the absence of nonlinear transfer functions applied to the cell state: the contribution from past states may remain unchanged over time.

Thanks to the context vector, the LSTM is able to keep in memory at each iteration information from any previous iteration.

Each LSTM network is trained by feeding several days of training data to the network and adjusting weights associated with each layer so that an objective error function E on the output is minimized. This minimization process corresponds to the training period and can be performed using backpropagation with a gradient decent method or a stochastic gradient descent, for instance in D. Kingma and J. Ba. Adam: A method for stochastic optimization. arXiv preprint arXiv:1412.6980, 2014.

LSTM requires that many hyperparameters are set during the training period, for instance learning rate (rate for changing weights), length of the input sequence, time span between each step in the input sequence, number of neurons contained in a layer, quantity of information that should be forgotten in the forget layer of an LSTM cell.

In the case of glycemia prediction, LSTM neural network may be run with a reduced number of input signals, with a reduced number of layers inside the cell, and still produce glycemia predictions of good quality.

Using LSTM neural network and the Pharmacokinetic pre-processing step, a glycemia prediction system featuring shorter data collection, fewer and shorter training periods, shorter calculation time to predict is able to ensure:
a glycemia level and a Root Mean Square Error (RMSE) of the predicted glycemia for a prediction time of 30 minutes compared to the temporally corresponding measured glycemia is below 30 mg/dL, preferably below 20 mg/dL and even more preferably below 10 mg/dL.
a glycemia level and a Root Mean Square Error (RMSE) of the predicted glycemia for a prediction time of 90 minutes compared to the temporally corresponding measured glycemia is below 50 mg/dL, preferably below 40 mg/dL and even more preferably below 30 mg/dL.

Another aspect of the invention relates to a method for recommending a quantity of insulin to inject or a sugar intake to ingest to reach a target glycemia at a certain horizon.

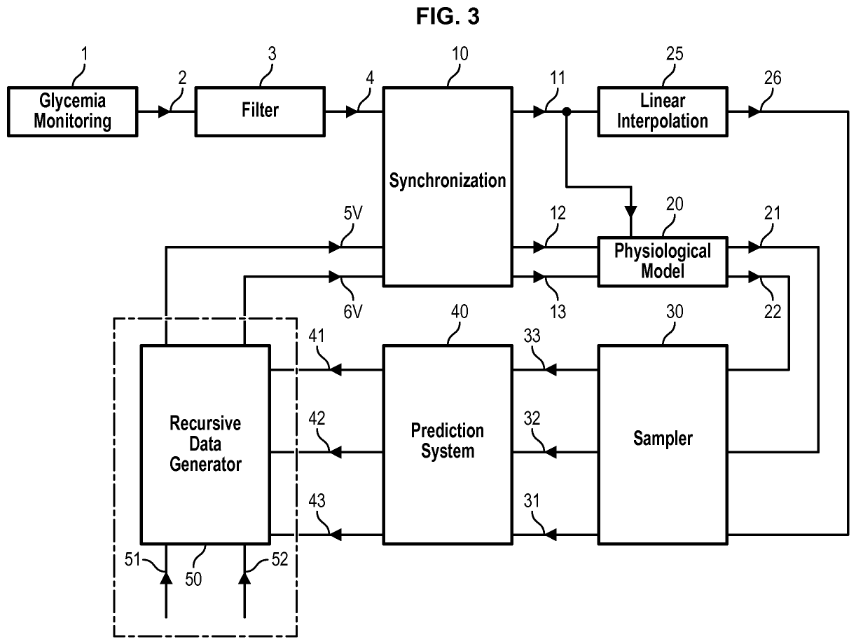

An example of the glycemia recommending method is described on FIG. 3.

Most of the figure is identical to FIG. 1, and the corresponding parts have already been presented above.

The differences are framed by dotted lines.

The prediction system 40 comprises a plurality of different neural networks arranged in parallel.

The prediction system thus delivers a plurality of different glycemia predictions 41, 42 and 43, corresponding to a plurality of different horizons.

The plurality of predictions is sent into a recursive data generator 50.

The recursive data generator 50 is able to generate virtual data set of dated discrete insulin quantity to inject 5V into the subject and virtual data set of dated discrete food quantity to ingest 6V by the subject.

The virtual data set of dated discrete food quantity to ingest 6V by the subject includes a quantity of glucose, but may also include other types of food such as lipids, free fatty acids, proteins and fibers

The recursive data generator 50 may also be able to generate virtual data set of dated level of physical exercise intensity to perform by the patient.

The two virtual data sets 5V and 6V substitute to the previous signals 5 and 6 of FIG. 1, and are sent into the synchronization device 10.

The virtual set of dated level of physical exercise intensity to perform by the patient may also be sent into the synchronization device 10.

The recursive data generator 50 features a first entry to retrieve a target glycemia interval 51 to be reached at a certain horizon, and a second optional entry of the data generator to retrieve a constraint 52.

The constraint 52 may be a maximum duration of time above a glycemia level not to exceed, a minimum glycemia level below which not to fall, a stable glycemia level after a certain time, or a minimum insulin quantity to inject, a wish to perform a physical exercise . . . . When a target glycemia interval 51 to be reached at a certain horizon is received by the recursive data generator, the recursive data generator 50 engenders two virtual data sets 5V and 6V, and optionally a virtual set of dated level of physical exercise intensity to perform by the patient.

The prediction system produces then a plurality of glycemia predictions 41, 42 and 43. According to these predictions and the target glycemia interval 51 to be reached at a certain horizon, the recursive data generator 50 engenders modified data sets 5V, 6V and optionally a modified data set of dated level of physical exercise intensity to perform by the patient, in order to bring closer the glycemia predictions to the target glycemia.

A loop is consequently set in operation until the glycemia predictions match the target glycemia.

The final data sets 5V and 6V and optionally the final data set of dated level of physical exercise intensity to perform by the patient, correspond to recommendations of insulin to inject and/or food intake to ingest presented to the patient.

Optionally, a constraint 52 may be taken into account during the recursive loop so that glycemia predictions and/or final recommendations do not come into conflict with the constraint.

A method for treating type 1 diabetes is proposed comprising the following steps:
Retrieving a target glycemia interval (51) to be reached at a certain future time t+Δt;
Retrieving a data set of glycemia (2) previously measured on a patient;
Choosing an initial and virtual quantity of insulin to inject in the patient;
Applying the glycemia prediction method as previously described to produce at least one predicted glycemia at time t+Δt;
Recursively determining a data set URi(t) (5V) of dated amount of insulin to inject in the patient that correspond to a predicted glycemia inside the target glycemia interval at time t+Δt,
Administering an effective amount of insulin to the patient, the effective amount of insulin being the recursively determined dates amount of insulin to inject.

An observatory study at the Montpellier University Hospital in France was conducted, and the glycemia prediction method as described above was carried out on a sample including 14 patients with type 1 diabetes.
8 women and 6 men participated in the study, with the following features:
   age: 51±15,
   type 1 diabetes duration: 26±17 years,
   body mass index: 24.5±3.8 kg/m²,
   HbA1c: 7.09%±0.82%,
   Treatment: insulin pump (11 participants) or multiple daily insulin injections (3 participants),
   they all volunteered to track glycemia or blood glucose level using glucose monitoring by Abbott FreeStyle Libre (12 participants), Medtronic Enlite (1 participant) or Dexcom G4 (1 participant),
   they logged manually meal intakes and insulin doses for 30 days.

The study ran over 30 days, and no specific intervention on the usual diabetes treatment was undertaken during this period.

Based on measured data, predictive algorithms were trained to estimate future blood glucose fluctuations, with 30-, 60- and 90-minutes prediction horizons.

Accuracy of blood glucose levels predictors was evaluated using the Parkes Error Grid Analysis (EGA). Parkes error grid breaks down a scatterplot of reference glucose readings and evaluated glucose reading into five regions:
Zone A: Clinically accurate—No effect on clinical action;
Zone B: Little to no effect on clinical outcome;
Zone C: Likely to affect clinical outcome;
Zone D: Could have significant medical risk;
Zone E: Could have dangerous consequences.

The results of the glycemia prediction method evaluated using Mean Absolute Deviation (MAD), Mean Absolute Relative Deviation (MARD) and matching zones A+B in the EGA for 30-, 60- and 90-minutes prediction horizons are the following
30-minutes prediction horizon (mean±standard deviation):
   MAD=10.67 mg/dl±2.05 mg/dl
   MARD=6.98%±1.30%
   Parkes EGA A+B=99.93%±0.13%
60-minutes prediction horizon:
   MAD=22.73 mg/dl±5.49 mg/dl
   MARD=14.78%±3.25%
   Parkes EGA A+B=98.56%±1.00%
90-minutes prediction horizon:
   MAD=31.76 mg/dl±7.53 mg/dl
   MARD=20.78%±4.08%
   Parkes EGA A+B=96.29%±2.15%

1. Method for predicting glycemia of a patient including the steps of:
Retrieving a data set (2) of dated glycemia measured up to the time t on the patient;
Retrieving a quantity of insulin (5) injected in the patient;
Retrieving a quantity of glucose (6) ingested by the patient;
Generating, by a pharmacokinetic pre-processing device (20), an insulin blood concentration signal (21) and a carbs rate of appearance signal (22), from the glycemia data set (2), the quantities of injected insulin (5) and ingested glucose (6),
Predicting by at least one Neural Network at least one glycemia (41) at prediction time t+Δt from the glycemia data (2) and from the signals generated (21) (22), the neural network being a Recurrent Neural Network able to keep in memory at each iteration information from any previous iteration.

2. Method according to claim 1 further including the steps of:
Retrieving at least one type of food ingested by the patient and at least one corresponding quantity of food ingested, the type of food being chosen among lipids, free fatty acids, proteins and fibers;
Generating, by the pharmacokinetic pre-processing device (20), an insulin blood concentration signal (21), a carbs rate of appearance signal (22) and optionally a free fatty acid blood concentration signal, from the glycemia data set (2), the quantities of injected insulin (5) and ingested glucose (6) and at least one quantity of food ingested.

3. Method according to claim 1 or 2 further including the steps of:
Retrieving a level of physical and/or emotional activity from a user statement, a heart pulse monitoring system, a photoplethysmograph, an electrocardiograph or an electroencephalograph,
Predicting by at least one Recurrent Neural Network at least one glycemia at prediction time t+Δt from the glycemia data, the signals generated (21) (22) and the level of physical and/or emotional activity.

4. Method according to any of the claims 1 to 3, further including the steps of:
Retrieving at least one type of medication administered to the patient and at least one corresponding quantity of medication administered, the type of medication being chosen among Metformin, Sulfonylureas, Meglitinides, DPP-4 inhibitors, GLP-1 receptor agonists, SGLT2 inhibitors,
Predicting by at least one Recurrent Neural Network at least one glycemia at prediction time t+Δt from the glycemia data, the signals generated (21) (22) and at least one quantity of medication administered to the patient.

5. The method according to any of the claims 1 to 4, and further including the step of
Generating, by a plurality of Recurrent Neural Network arranged in parallel, a plurality of predicted glycemia corresponding to a plurality of prediction times, wherein at least one Recurrent Neural Network is of Long Short Term Memory type.

6. The method according to any of the claims 1 to 5 wherein a Root Mean Square Error (RMSE) of a predicted glycemia for a prediction time of 30 minutes compared to the temporally corresponding measured glycemia is below 30 mg/dL and wherein a Root Mean Square Error (RMSE) of a predicted glycemia for a prediction time of 90 minutes compared to the temporally corresponding measured glycemia is below 50 mg/dL.

7. The method according to any of the claims 1 to 6, wherein generating an insulin blood concentration signal (21), a carbs rate of appearance signal (22), and optionally a free fatty acid blood concentration signal, further includes the step of:
Resolving at least one second order system of equations for each signal.

8. The method according to claim 7, further including the steps of:
Retrieving amounts of rapid acting Uᵢ and long-acting U_{i,2} insulin injected in the patient;
Resolving the second order system of equations:
$$\{ \begin{matrix}{\dot{X}}_{2} = - {ω}_{i} ( {X}_{2} - {X}_{1} ) - {k}_{l} {X}_{2} ( t ) \\ {\dot{X}}_{1} = - {ω}_{i} ( {X}_{1} - {U}_{i} )\end{matrix}$$
Wherein
Uᵢ is the amount of rapid-acting insulin injected in the patient,
X₁ is the rapid-acting insulin concentration in a fictive intermediate compartment,
X₂ is the rapid-acting insulin blood concentration and
the parameter ωᵢ is the rapid-acting insulin transfer rate between compartments, and
the parameter kₗ represents irreversible degradation of insulin in the liver.
Resolving the second order system of equations
$$\{ \begin{matrix}{\dot{X}}_{2 , 2} = - {ω}_{i , 2} ( {X}_{2 , 2} - {X}_{1 , 2} ) - {k}_{l} {X}_{2 , 2} \\ {\dot{X}}_{1 , 2} = - {ω}_{i , 2} ( {X}_{1 , 2} - {U}_{i , 2} )\end{matrix}$$
Wherein
U_{i,2} is the amount of long-acting insulin injected in the patient,
X_{1,2} is the long-acting insulin concentration in a fictive intermediate compartment,
X_{2,2} is the long-acting insulin blood concentration and
the parameter ω_{i,2} is the long-acting insulin transfer rate between compartments, and
the parameter kₗ represents irreversible degradation of insulin in the liver.
Retrieving an amount of proteins ingested by the patient;
Adding a virtual amount of ingested glucose equal to 60% of the amount of protein ingested to form an effective amount Uᵣ of ingested glucose
Resolving the second order system of equations:
$$\{ \begin{matrix}{\dot{D}}_{2} = - {ω}_{r 2} ( {D}_{2} - {D}_{1} ) \\ {\dot{D}}_{1} = - {ω}_{r 1} ( {D}_{1} - {U}_{r} )\end{matrix}$$
Wherein
D₁ is the glucose concentration in a fictive intermediate compartment,
D₂ is the glucose blood concentration, and
the parameters ωᵣ₁ and ωᵣ₂ are glucose transfer rates between compartments.
Retrieving an amount U_{FFA} of free fatty acids ingested by the patient;
Resolving the second order system of equations:
$$\{ \begin{matrix}{\dot{F}}_{2} = - {ω}_{F F A} ( {F}_{2} - {F}_{1} ) \\ {\dot{F}}_{1} = - {ω}_{F F A} ( {F}_{1} - {U}_{F F A} )\end{matrix}$$
Wherein
F₁ is the free fatty acids concentration in a fictive intermediate compartment,
F₂ is the free fatty acids blood concentration and the parameter ω_{FFA} is the free fatty acids transfer rate between compartments.

9. The method according to any of the preceding claims further including the step of calibration of the pharmacokinetic pre-processing device, corresponding to the determination of the parameters involved in the second order systems of equations.

10. The method according to claim 9 wherein the step of calibration of the pharmacokinetic pre-processing device is accomplished through a hybrid method combining a Nelder-Mead algorithm and a Particle Swarm Optimization method, including the steps of:
Creating (100) an error function ƒ depending on the parameters involved in the second order systems of equations able to compare a predicted glycemia and a measured glycemia;
Generating (101) a first set of p parameters corresponding to classical values, where p is the number of parameters involved in the second order systems of equations;
Logging (102) the set of p parameters in the pharmacokinetic pre-processing device;
Running (103) the pharmacokinetic pre-processing device using historical user data to predict at least one glycemia;
Calculating the value of the error function ƒ depending on p parameters involved in the second order systems of equations;
Comparing (104) the value of the error function ƒ to a predetermined threshold;
According to the result of the comparison terminating (106) the method and keeping the first set of p parameters as the set of optimized parameters or else performing (105) an optimization process loop that includes the steps of:
Generating randomly a set of V vectors Xi of dimension p representing V sets of p parameters;
Generating for each of the V vectors Xi a trajectory Ti of successive vectors X_{i,n} each corresponding to a time step n, the transit from one vector of time step n to the following time step n+1 including the steps of:
Identifying (E1) X_{i best} corresponding to the minimum value of ƒ calculated among the vectors of the trajectory Ti corresponding to a maximum time step n;
Identifying (E2) X_{j≠i best} corresponding to the minimum value of ƒ calculated among the vectors of all trajectories but Ti and corresponding to a maximum time step n;
Determining (E3) a vector Y_{i,n+1} with the Particle Swarm Optimization method formula:
Y_{i,n+1}=c₁*X_{i,n}+c₂*[X_{i,best}−X_{i,n}]+c₃*[X_{j≠i best}−X_{i,n}]
wherein c₁, c₂, c₃ are parameters so that c₁=1.8
0≤c₂≤1.62 and 0≤c₃≤1.62;
Applying (E4) a Nelder-Mead algorithm step to each Y_{i,n+1}, in order to produce a vector X_{i,n+1} local minimum of the function ƒ using a simplex of (p+1) vectors ₘZ_{i,n+1}, with m∈ [symbol] 1,p+1 [symbol] , each vector of dimension p defined by ₘZ_{i,n+1}^{k}=Y_{i,n+1}^{k}(1+m*δ) with k∈ [symbol] 1,p [symbol] and δ=0.05, wherein Y_{i,n+1}^{k} is the k-th coordinate of the vector Y_{i,n+1} and ₘZ_{i,n+1}^{k} is the k-th coordinate of the vector ₘZ_{i,n+1};
Comparing (E5) the value of ƒ(X_{i,n+1}) and a predetermined threshold;
Repeating steps (E1) to (E5) until a value of ƒ(X_{i,n+1}) falls below the predetermined threshold or else terminating (107) the method terminating the method, the corresponding X_{i,n+1} being the set of optimized parameters.

11. The method according to any claims 9 to 10 further including a step of offering to the user to realize short and simple experiences in order to retrieve the corresponding data.

12. Method for recommending a food quantity to including the steps of:
Retrieving a target glycemia interval (51) to be reached at a certain future time t+Δt;
Retrieving a data set of glycemia (2) previously measured on a patient;
Choosing an initial and virtual quantity of food to ingest by the patient;
Applying the glycemia prediction method according to one of the claims 1 to 10 to produce at least one predicted glycemia at time t+Δt;
Recursively determining a data set URf(t) (6V) of dated food quantity to ingest by the patient that correspond to a predicted glycemia inside the target glycemia interval at time t+Δt,
Displaying a message to the patient mentioning the determined food quantity to ingest.

13. A method according to claim 12 further including the steps of
Retrieving a constraint (52) possibly containing:
a maximum duration of time above a glycemia level not to exceed;
a minimum glycemia level below which not to fall;
a wish to perform a physical exercise;
Choosing an initial and virtual level of physical intensity;
Recursively determining the data set URf(t) and a data set URa(t) of dated level of physical exercise intensity to perform by the patient so that:
predictions of glycemia by the prediction system do not exceed the glycemia level longer than the maximum duration of time
predictions of glycemia by the prediction system all stand above the minimum glycemia level,
URa(t) is compatible with the wish to perform a physical exercise
Displaying the corresponding recommendation to the patient.

14. Method for recommending an insulin quantity to inject including the steps of:
Retrieving a target glycemia interval (51) to be reached at a certain future time t+Δt;
Retrieving a data set of glycemia (2) previously measured on a patient;
Choosing an initial and virtual quantity of insulin to inject in the patient;
Applying the glycemia prediction method according to one of the claims 1 to 10 to produce at least one predicted glycemia at time t+Δt;
Recursively determining a data set URi(t) (5V) of dated amount of insulin to inject in the patient that correspond to a predicted glycemia inside the target glycemia interval at time t+Δt,
Displaying a message to the patient mentioning the determined amount of insulin to inject.

15. A method according to claim 14 further including the steps of
Retrieving a constraint (52) possibly containing:
a maximum insulin quantity to inject;
a maximum duration of time above a glycemia level not to exceed;
a minimum glycemia level below which not to fall;
a wish to perform a physical exercise;
Choosing an initial and virtual level of physical intensity;
Recursively determining the data set URi(t) and a data set URa(t) of dated level of physical exercise intensity to perform by the patient so that:
predictions of glycemia by the prediction system do not exceed the glycemia level longer than the maximum duration of time
predictions of glycemia by the prediction system all stand above the minimum glycemia level,
URi(t) is compatible with the maximum quantity of insulin to inject
URa(t) is compatible with the wish to perform a physical exercise
Displaying the corresponding recommendation to the patient.

16. Insulin for use in the treatment of type 1 diabetes characterised in that the dosage regimen is determined by a method including the steps of:
Retrieving a target glycemia interval (51) to be reached at a certain future time t+Δt;
Retrieving a data set of glycemia (2) previously measured on a patient;
Choosing an initial and virtual quantity of insulin to inject in the patient;
Applying the glycemia prediction method according to one of the claims 1 to 10 to produce at least one predicted glycemia at time t+Δt;
Recursively determining a data set URi(t) (5V) of dated amount of insulin to inject in the patient that correspond to a predicted glycemia inside the target glycemia interval at time t+Δt,
Displaying a message to the patient mentioning the determined amount of insulin to inject.

17. A glycemia prediction device comprising:
a glycemia monitoring system (1) able to measure the glycemia of the patient and transfer data of dated glycemia measures of the patient,
a first device able to retrieve and transfer data of dated insulin quantity injected in the patient,
a second device able to retrieve and transfer data of dated food type and quantity ingested by the patient,
an optional third device able to retrieve and transfer data of dated physical and/or emotional activity of the patient
a pharmacokinetic pre-processing device (20) linked to the glycemia monitoring system, to the first, second and optional third devices, able to generate from the data sets of dated measured glycemia (2), dated insulin quantity injected (5) and dated food type and quantity ingested an insulin blood concentration signal (12), a carbs rate of appearance signal (13), and optionally a free fatty acid blood concentration signal.
a prediction system (40) comprising at least one Long Short Term Memory neural network and linked to the pharmacokinetic pre-processing device (20), able to produce from the data set of dated measured glycemia, optionally dated physical and/or emotional activity and the signals generated (12) (13) at least one predicted glycemia (41).

18. A glycemia recommendation device comprising:
a glycemia prediction device according to claim 16,
a recursive data generator (50) linked to the output, to the first, second and optional third devices of the glycemia prediction device and able to create and modify according to the output of the glycemia prediction device a virtual data set of dated discrete insulin quantity to inject into the patient, a virtual data set of dated discrete food quantity to ingest by the patient, and an optional virtual data set URa(t) of dated level of physical exercise intensity to perform by the patient
a first entry of the data generator to retrieve a target glycemia interval (51) to be reached at a certain horizon,
a second optional entry of the data generator to retrieve a constraint (52) possibly containing
a maximum insulin quantity to inject;
a maximum duration of time above a glycemia level not to exceed;
a minimum glycemia level below which not to fall;
a wish to perform a physical exercise;
a displayer to present the recommendation to the patient
